(19) United States
(12) Patent Application Publication
(10) Pub. No.: US 2018/0059128 A1
(43) Pub. Date: Mar. 1, 2018

(54) HOME TEST KIT FOR PREPARING AN EXTRACT FROM A HERB

(71) Applicant: Keystone Labs Inc., Edmonton (CA)

(72) Inventors: Jodi Colleen McDonald, Edmonton (CA); Roderick James Szarka, Edmonton (CA)

(21) Appl. No.: 15/690,949

(22) Filed: Aug. 30, 2017

Related U.S. Application Data
(60) Provisional application No. 62/381,158, filed on Aug. 30, 2016.

(51) Int. Cl. G01N 33/94; G01N 1/40

(52) U.S. Cl. CPC G01N 33/948; G01N 1/4077; G01N 2001/4061; G01N 2001/4088; G01N 1/4055

(57) ABSTRACT

A home test kit is provided for preparing an extract from herbs, in particular, extracts from medical marijuana that can be sent into an independent lab for testing the presence and concentration of cannabinoids.

### CROSS-REFERENCE TO RELATED APPLICATIONS

This application claims the benefit of U.S. Patent Application Ser. No. 62/381,158 filed Aug. 30, 2016, which is incorporated by reference in its entirety.

### TECHNICAL FIELD

The present disclosure is related to the field of home test kits for preparing an extract from herbs, in particular, extracts from medical marijuana that can be sent into an independent lab for testing the presence and concentration of cannabinoids.

### BACKGROUND

Many patients who have been prescribed medical marijuana as a means to treat their disease or ailment grow their own marijuana, as opposed to obtaining the medical marijuana from an authorized provider. The problem with this situation is the chemical structure of the home-grown marijuana is unknown as to what cannabinoids are present along with their concentration. An authorized provider can have access to lab facilities that enable the analysis of a particular strain of medical marijuana to determine the presence and concentration of cannabinoids, which the patient growing their own medical marijuana likely does not.

Not knowing what cannabinoids are present in the patient's home-grown marijuana, and their strength or concentration, is problematic as it can be a “hit or miss” proposition in whether the strength of the therapeutic cannabinoids in the home-grown marijuana required by the patient is too little or too much.

For the patient to simply send samples of their home-grown marijuana to a lab for independent testing is problematic as the simple act of mailing samples of the home-grown marijuana to a lab for testing can be contrary to laws concerning the trafficking of controlled substances in the jurisdiction where the patient resides. In Canada, for example, the Controlled Drugs and Substances Act, S.C. 1996, c. 19 prohibits the transport of marijuana except by a licensed producer who is authorized under Health Canada regulations to do so.

For the purposes of this specification, it is presumed that the patient has a doctor's prescription for medical marijuana to treat a diagnosed disease or ailment, and that the patient also has authorization to possess medical marijuana, and further has authorization to grow their own medical marijuana.

It is, therefore, desirable to provide a home test kit that enables a patient to obtain an extract of the patient's medical marijuana, which can then be sent by the patient to an independent lab to test the cannabinoids in the patient's home-grown medical marijuana.

### SUMMARY

In some embodiments, a home test kit can be provided for preparing an extract from herbs, in particular, extracts from medical marijuana that can be sent into an independent lab for testing the presence and concentration of cannabinoids of the medical marijuana.

Broadly stated, in some embodiments, a home test kit can be provided for preparing an extract from a herb, the kit comprising: a first vial with a removable first lid, the first vial comprising an extracting fluid disposed therein, the first vial further comprising a chemical marker disposed therein; a second vial with a removable second lid; a syringe; and a filter configured to operatively coupled to the syringe.

Broadly stated, in some embodiments, the home test kit can comprise one or more weighing papers.

Broadly stated, in some embodiments, the home test kit can further comprise a measuring spoon.

Broadly stated, in some embodiments, the home test kit can further comprise a packing tool.

Broadly stated, in some embodiments, the home test kit can further comprise a foam insert configured for holding the first and second vials in an upright position.

Broadly stated, in some embodiments, the home test kit can further comprise a mailing box.

Broadly stated, in some embodiments, the home test kit can further comprise a set of instructions.

Broadly stated, in some embodiments, a method can be provided for preparing an extract from a herb, the method comprising the steps of: grinding an amount of the herb into a powder; placing a predetermined amount of the powder into a first vial comprising a removable first lid, the first vial comprising an extracting fluid disposed therein, the first vial further comprising a chemical marker disposed therein; placing the first lid on the first vial, and shaking the first vial at least once for a predetermined amount of time; withdrawing the fluid in the first vial after the herb contained in the first vial has settled to the bottom of the first vial; and passing the withdrawn fluid through a filter into a second vial comprising a removable second lid.

Broadly stated, in some embodiments, the step of shaking the first vial can be performed more than once.

Broadly stated, in some embodiments, the method can further comprise the step of placing the second lid on the second vial.

Broadly stated, in some embodiments, the extracting fluid can comprise a predetermined amount of one or more of methanol and chloroform.

Broadly stated, in some embodiments, the chemical marker can comprise a predetermined amount of one or more of prazepam, lorazepam and any other form of benzodiazepines.

### BRIEF DESCRIPTION OF THE DRAWINGS

FIG. 1 is a perspective view depicting one embodiment of a home test kit for preparing an extract from a herb with the components of the test kit nested in a packing foam insert placed in a mailing box.

FIG. 2 is a perspective view depicting the home test kit of FIG. 1 with the components of the test kit removed from the packing foam insert.

FIG. 3 is a perspective view depicting the placement of an amount of dried herb onto a weighing paper.

FIG. 4 is a perspective view depicting the grinding of the dried herb of FIG. 3 with another weighing paper placed on top thereof.

FIG. 5 is a perspective view depicting an open vial containing extracting fluid placed in the foam insert of FIG. 1.

FIG. 6A is a perspective view depicting an amount of the ground dried herb of FIG. 4 into a measuring spoon.

FIG. 6B is a perspective view depicting the packing of the dried herb into the measuring spoon of FIG. 6A.

FIG. 6C is a perspective view depicting the step of leveling the dried herb in the measuring spoon of FIG. 6B.

FIG. 6D is a perspective view depicting the dried herb in the measuring spoon of FIG. 6C being leveled.

FIG. 7A is a perspective view depicting placing the measuring spoon of FIG. 6D over a third weighing paper.

FIG. 7B is a perspective view depicting the placement of dried herb on the third weighing paper of FIG. 7A.

FIG. 7C is a perspective view depicting the placement of dried herb in a crease on the third weighing paper of FIG. 7B.

FIG. 7D is a perspective view depicting the dried herb of FIG. 7C being lifted.

FIG. 8 is a perspective view depicting the pouring of the dried herb of FIG. 7D into the open vial of FIG. 5.

FIG. 9 is a perspective view depicting the shaking of the vial of FIG. 8 after being capped with its lid.

FIG. 10 is a perspective view depicting the vial of FIG. 9 placed in the foam insert of FIG. 1 to allow the herb to settle therein.

FIG. 11A is a perspective view depicting the placement of a needle onto a syringe.

FIG. 11B is a perspective view depicting the syringe of FIG. 11A with the needle placed thereon.

FIG. 12 is a perspective view depicting the syringe of FIG. 11B withdrawing fluid from the vial of FIG. 10.

FIG. 13 is a perspective view depicting the placement of a filter onto the syringe of FIG. 12.

FIG. 14 is a perspective view depicting the passing of the fluid in the vial of FIG. 10 through the filter of FIG. 13 into an empty vial.

FIG. 15 is a perspective view depicting the placement of the vial of FIG. 14 containing the filtered fluid, after being capped with its lid, into the foam insert of FIG. 1.

FIG. 16 is a perspective view depicting the placement of a mailing label onto the mailing box of FIG. 1 containing the capped vial of filtered fluid.

FIG. 17 is a perspective view depicting the placement of a tamperproof label onto the mailing box of FIG. 16.

FIG. 18 is a top plan view depicting one embodiment of an unfolded mailing box of the home test kit of FIG. 1.

### DETAILED DESCRIPTION OF EMBODIMENTS

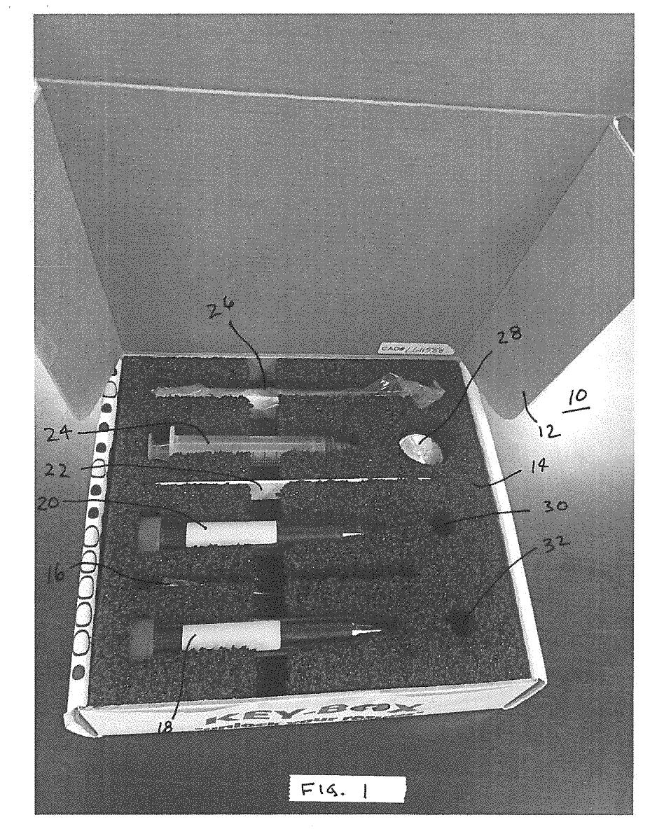

Referring to FIGS. 1 and 2, one embodiment of home test kit 10 is shown. In some embodiments, home test kit 10 can comprise mailing box 12 having foam insert 14 disposed therein. Home test kit 10 can further comprise a first vial 18, further comprising a removable lid, wherein first vial 18 can comprise an extracting fluid, such as a reagent, and can further comprise a chemical marker disposed therein. In some embodiments, the extracting fluid can comprise a predetermined volume and concentration of one or more of methanol and chloroform, or any other reagent well known to those skilled in the art that can produce a liquid extract from a herb. In a representative embodiment, the reagent can comprise 10 mL of neat methanol.

In some embodiments, the chemical marker can comprise a predetermined volume and concentration of one or more of prazepam, lorazepam, and any other form benzodiazepines, or any other chemical marker well known to those skilled in the art that will not interfere with the reagent producing a liquid extract from a herb. In a representative embodiment, the chemical marker can comprise 0.1 mL of one or more of prazepam, lorazepam and any other form of benzodiazepines having a concentration of 50 μg/mL to 100 μg/mL. For the purposes of this description, the term “benzodiazepine” shall mean any chemical compound in the class of chemical compounds comprising a benzene ring and a diazepine ring, as well known to those skilled in the art.

In some embodiments, home test kit 10 can comprise second vial 20, having a removable lid; syringe tube 24, syringe needle 16, packing tool 22, measuring spoons 26 and syringe filter 28, all of which can be fitted into dedicated openings therefor disposed in foam insert 14. Foam insert 14 can also comprise openings 30 and 32 disposed therein for receiving and supporting vials 18 and 20 in an upright working position. In some embodiments, one or both of vials 18 and 20 can comprise a 15 ml conical vial.

Referring to FIG. 18, one embodiment of mailing box 12 is shown, wherein the illustrated embodiment of box 12 can be formed from a single piece of cardboard or any such other material well known to those skilled in the art for forming a mailing box.

In some embodiments, home test kit 10 can comprise one or more pieces of weighing paper (not shown), and a set of instructions for use by the patient explaining to prepare an extract of the patient's home-grown medical marijuana (one embodiment of which is attached hereto as Appendix “A”).

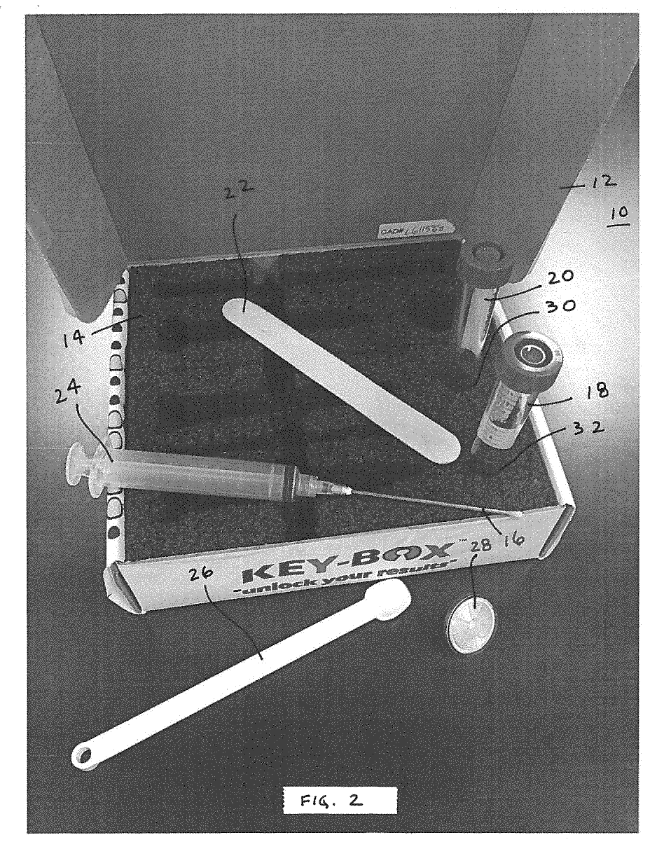

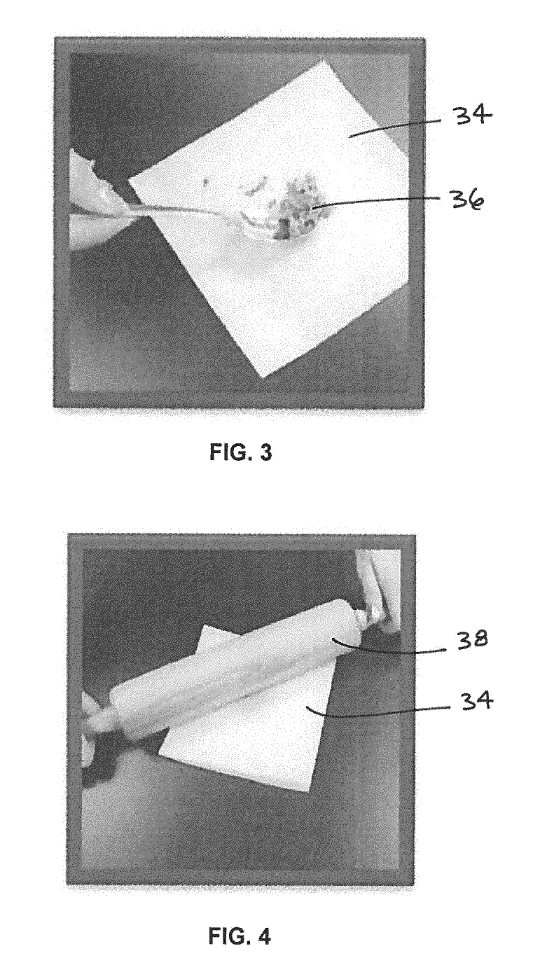

To prepare a liquid extract from a dried herb, such as dried marijuana used for medical purposes, an amount of the dried herb 36 can be placed on a piece of weighing paper 34, such as a piece of 6 inch by 6 inch weighing paper, as shown in FIG. 3. This amount can be approximately one to two teaspoons. Then, a second piece of weighing paper can be placed on top of the dried herb.

Referring to FIG. 4, dried herb 36 sandwiched between two weighing papers 34 to grind the dry herb until it is a fine powder, such as with or a heavy plastic or metal cylinder or cup, or with rolling pin 38. The finer the dry herb, the more accurate the weight of the ground dry herb sample for testing can be, which can allow for more accurate results. In other embodiments, a mortar and pestle can be used to grind the dried herb, as described and illustrated in the attached Appendix “A”.

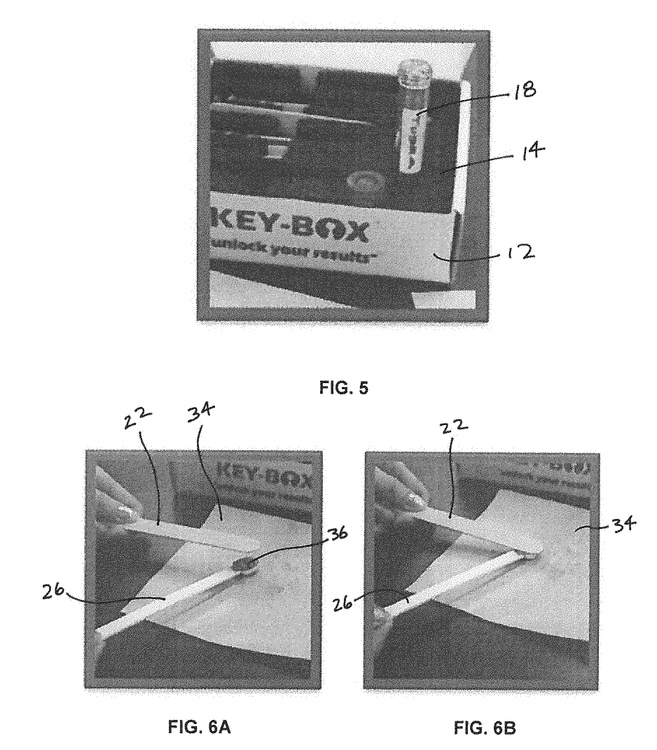

In some embodiments, vial 18, containing the extracting fluid and chemical marker therein, can be placed into hole 30 or hole 32 provided in foam insert 14, so that vial 18 is vertical, as shown in FIG. 5. The cap can be removed from vial 18 and set aside temporarily.

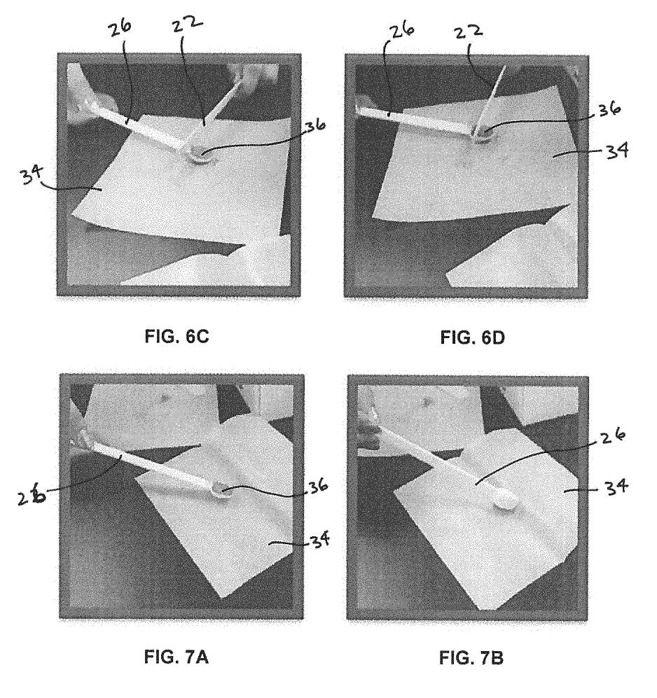

Referring to FIGS. 6A to 6D, measuring spoon 26 can be used to scoop up the ground dry herb so that it is heaping. In a representative embodiment, spoon 26 can comprise a 1.25 ml measuring spoon. Packing tool 22, which can be a 6 inch×¾ inch wooden stick (such as a tongue depressor), can then be used to lightly pack the ground dry herb into the spoon. Afterwards, a straight edge of packing tool 22 can then be used to scrape off the excess dry herb so that it is level with the top of spoon 26.

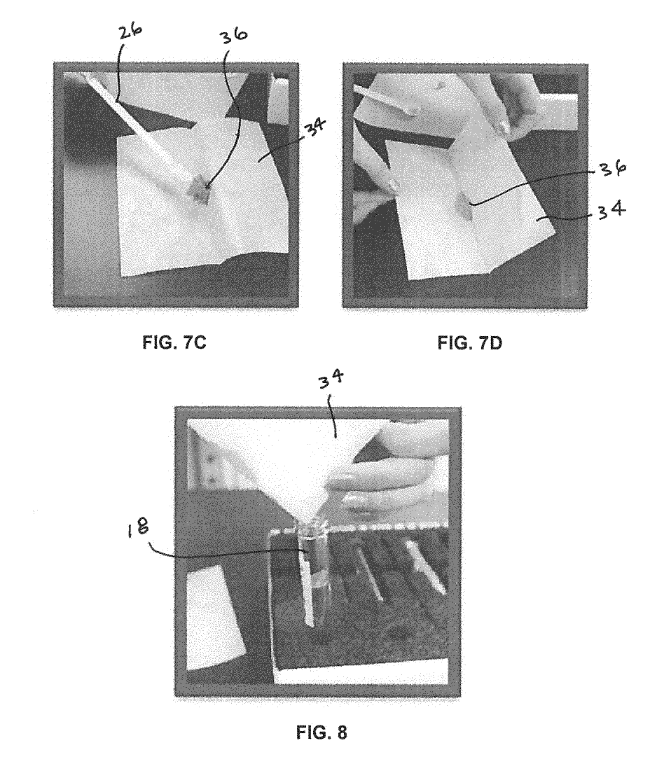

Referring to FIGS. 7A to 7D, the measured herb from the spoon can then be poured onto a third weighing paper. If required, spoon 26 can be flicked or tapped to remove any extra ground herb stuck to the spoon surface. The third weighing paper can then be picked up by the opposite sides of the weighing paper with the ground herb. The weighing paper will be in the shape of a “U”. A crease can be made in one end of the weighing paper, which can then be placed into the open mouth of vial 18, the one filled with the extracting liquid. The measured dry herb from the weighing paper can then be poured into vial 18, as shown in FIG. 8. The lid for vial 18 can then be placed back on, and then twisted to secure the lid tightly to vial 18.

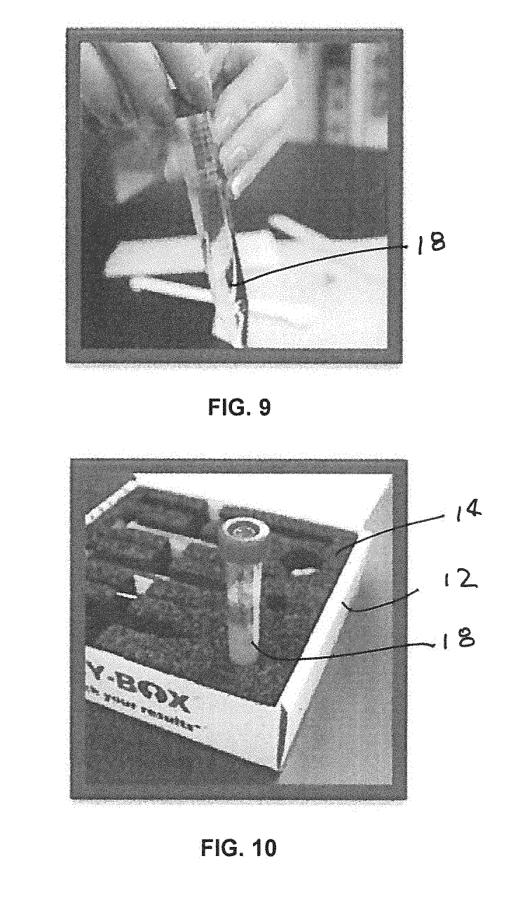

In some embodiments, vial 18, now filed with the herb and the extracting fluid, can be shaken for a predetermined length of time as shown in FIG. 9, and then placed back into hole 30 or 32 in foam insert 14, as shown in FIG. 10. In a representative embodiment, vial 18 can be shaken for approximately 30 seconds. At this point, the herb can be allowed to settle for another period of time. In a representative embodiment, the settling period can be approximately 2 minutes. In some embodiments, this shaking and settling process can be repeated. In a representative embodiment, this process can be repeated 9 more times for a total of 10 times.

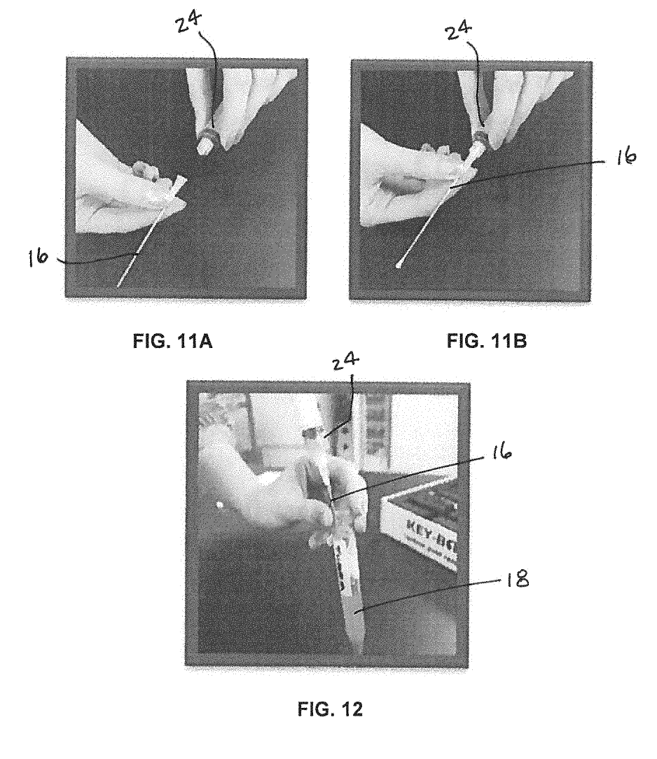

Once the shaking and settling cycles have been completed, syringe needle 16 can be placed on syringe tube 24, as shown in FIGS. 11A and 11B. In some embodiments, syringe needle 16 can screw onto the end of syringe tube 24. Once all of the herb has settled to the bottom of vial 18, the end of syringe needle 16 can be placed into vial 18 to withdraw the liquid into syringe tube 24 by pulling up on its plunger in a manner so as to not disturb the settled herb, as shown in FIG. 12.

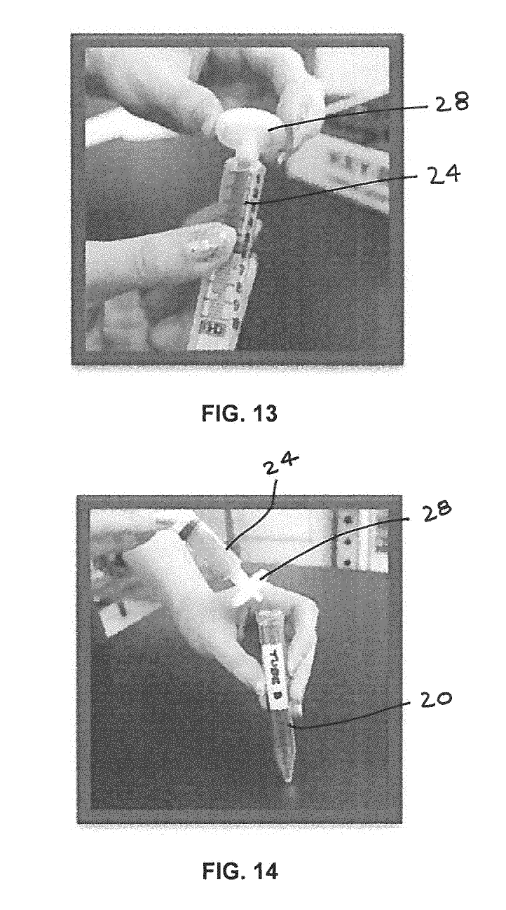

In some embodiments, syringe needle 16 can then be twisted off of syringe tube 24, followed by twisting syringe filter 28 onto syringe tube 24, as shown in FIG. 13. Vial 20 can then be placed in the remaining open hole 30 or 32 in foam insert 14 and then have its lid removed. At this point, the end of syringe filter 28 can be placed into the opening of vial 20 to receive liquid forced through it by depressing the plunger of syringe 24, as shown in FIG. 14. In some embodiments, syringe filter 28 can comprise a 0.45 um filter element to prevent any particles greater than 0.45 um in size from passing through. Once all of the liquid in syringe tube 24 has been dispensed through syringe filter 28 into vial 20, the lid of vial 20 can be placed on vial to secure and seal the filtered liquid therein.

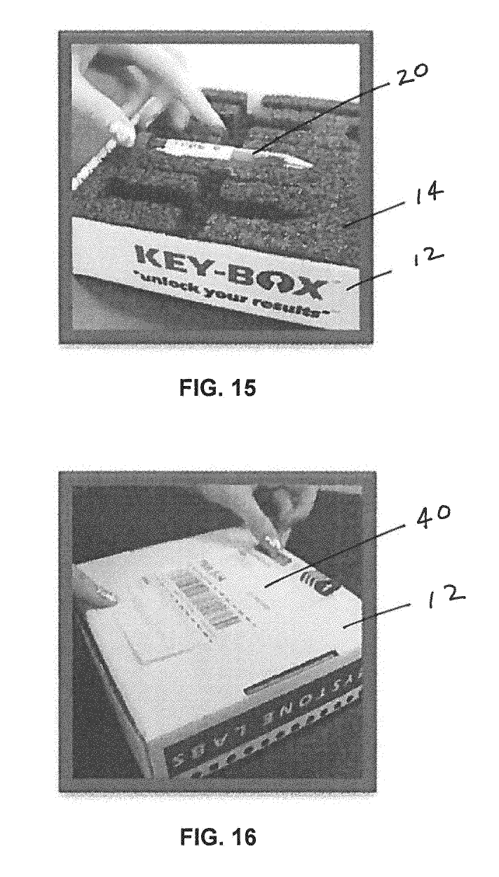

At this point, vial 20 can be placed back into its horizontal position in foam insert 14 before closing mailing box 12, as shown in FIG. 15. A mailing label can then be placed on mailing box 12 to enable it to be mailed to an independent lab for testing the cannabinoids in the filtered liquid extract in vial 20, as shown in FIG. 16.

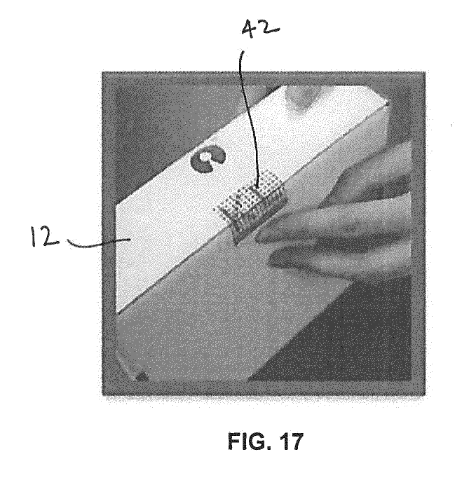

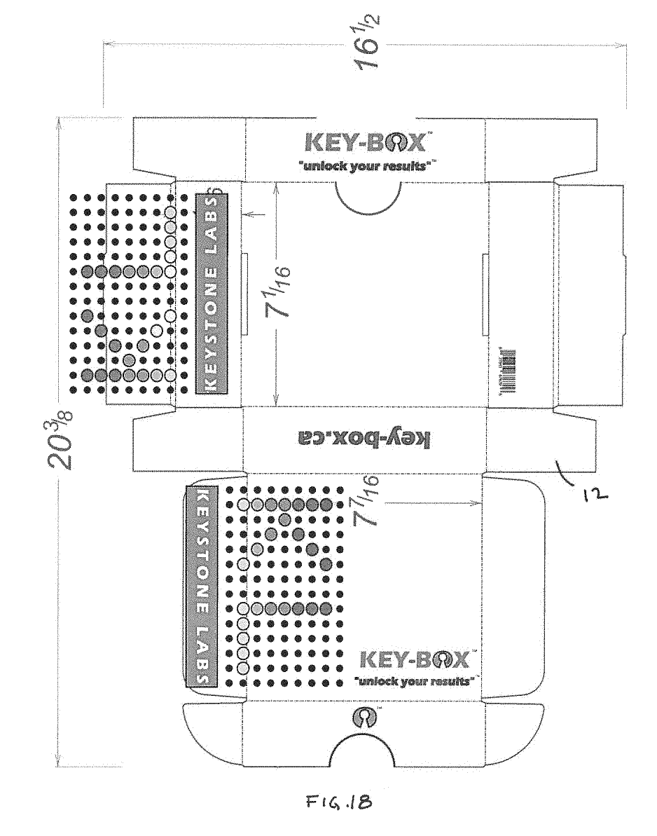

In some embodiments, a tamperproof label can be placed over an edge of mailing box 12 so that the lab receiving mailing box 12 can visually ensure that mailing box 12 has not been tampered with after being mailed by the patient, as shown in FIG. 17.

When the herb extract sample is received by the lab, they can test for the presence of the chemical marker as well as test for the presence and concentration of cannabinoids in the extract sample. The tests and techniques to test for the presence of cannabinoids are well known to those skilled in the art.

In some embodiments, the chemical marker disposed in vial 18 along with the extract sample can also provide information to the independent lab testing the extract sample. First, by testing for the mere presence of the chemical marker, the lab can affirm the authenticity of the extract sample to the extent that the extract sample was prepared using the materials originally packaged in home test kit 10. If the testing of the extract sample indicates no presence of the chemical marker, then the lab can deduce that the extract sample was not prepared with the materials originally packaged with the home test kit. The lack of the chemical marker being present in the extract sample could indicate that the patient did not use the materials in the home test kit as originally packaged, and simply used another source of an extracting fluid, or it could indicate that the extract sample was tampered with or exchanged with another extract sample produced with an extracting fluid not containing the chemical marker. In either case, the independent lab can conclude that the extract sample has been compromised and, thus, cannot be tested any further. The independent lab can then advise the patient of this finding so that another extract sample can be prepared using another home test kit in accordance with the instructions as provided.

In some embodiments, the lab testing the extract sample may identify the presence of the chemical marker, but not at the concentration that was originally packaged in vial 18. In this instance, the patient may have spilled some of the extracting fluid and chemical marker in vial 18 in the process of the preparing the extract and may have simply topped vial 18 with additional extracting fluid that does not contain any chemical marker. Or this could indicate that the extract sample was exchanged or otherwise tampered with. In either case, the independent lab can conclude that the extract sample has been compromised and, thus, cannot be tested any further. The independent lab can then advise the patient of this finding so that another extract sample can be prepared using another home test kit in accordance with the instructions as provided.

In some embodiments, the chemical marker can be selected at concentration that is unique to a particular lab. Thus, the presence and concentration of a particular chemical marker can be used as a unique identifier of the lab who prepared the home test kit.

Although a few embodiments have been shown and described, it will be appreciated by those skilled in the art that various changes and modifications can be made to these embodiments without changing or departing from their scope, intent or functionality. The terms and expressions used in the preceding specification have been used herein as terms of description and not of limitation, and there is no intention in the use of such terms and expressions of excluding equivalents of the features shown and described or portions thereof, it being recognized that the invention is defined and limited only by the claims that follow.

1. A home test kit for preparing an extract from a herb, the kit comprising:
a) a first vial with a removable first lid, the first vial comprising an extracting fluid disposed therein, the first vial further comprising a chemical marker disposed therein;
b) a second vial with a removable second lid;
c) a syringe; and
d) a filter configured to operatively coupled to the syringe.

2. The home test kit as set forth in claim 1, further comprising one or more weighing papers.

3. The home test kit as set forth in claim 1, further comprising a measuring spoon.

4. The home test kit as set forth in claim 1, further comprising a packing tool.

5. The home test kit as set forth in claim 1, further comprising a foam insert configured for holding the first and second vials in an upright position.

6. The home test kit as set forth in claim 1, further comprising a mailing box.

7. The home test kit as set forth in claim 1, further comprising a set of instructions.

8. The home test kit as set forth in claim 1, wherein the extracting fluid comprise a predetermined amount of one or more of methanol and chloroform.

9. The home test kit as set forth in claim 1, wherein the chemical marker comprises a predetermined amount of one or more of prazepam, lorazepam and a benzodiazepine.

10. A method for preparing an extract from a herb, the method comprising the steps of:
a) grinding an amount of the herb into a powder;
b) placing a predetermined amount of the powder into a first vial comprising a removable first lid, the first vial comprising an extracting fluid disposed therein, the first vial further comprising a chemical marker disposed therein;
c) placing the first lid on the first vial, and shaking the first vial at least once for a predetermined amount of time;
d) withdrawing the fluid in the first vial after the herb contained in the first vial has settled to the bottom of the first vial; and
e) passing the withdrawn fluid through a filter into a second vial comprising a removable second lid.

11. The method as set forth in claim 10, wherein the step of shaking the first vial is performed more than once.

12. The method as set forth in claim 10, further comprising the step of placing the second lid on the second vial.

13. The method as set forth in claim 10, wherein the extracting fluid comprise a predetermined amount of one or more of methanol and chloroform.

14. The method as set forth in claim 10, wherein the chemical marker comprises a predetermined amount of one or more of prazepam, lorazepam and a benzodiazepine.
